(19) United States
(12) Patent Application Publication
(10) Pub. No.: US 2024/0350138 A1
(43) Pub. Date: Oct. 24, 2024

(54) Abdominal Tissue Lifting Device

(71) Applicant: Stetrix, Inc., Bartlett, TN (US)

(72) Inventors: David D. Blurton, Whiteville, TN (US); Mark Buchanan, Atoka, TN (US)

(21) Appl. No.: 18/637,870

(22) Filed: Apr. 17, 2024

Related U.S. Application Data
(60) Provisional application No. 63/497,339, filed on Apr. 20, 2023.
(60) Provisional application No. 63/503,060, filed on May 18, 2023.

(51) Int. Cl. A61B 17/08; A61B 17/00

(52) U.S. Cl. CPC A61B 17/085; A61B 2017/00473; A61B 2017/081; A61B 2560/04

(57) ABSTRACT

A hand held tissue lifting device comprises a flexible anchor including an upper surface and a lower surface, with a layer of adhesive on the lower surface for attachment to skin of a patient. A handle that is sized and configured to be hand held by a user is rotatably attached to the upper surface of the anchor. A cutout may be formed into the periphery of the anchor to keep the navel area open upon attachment of device to the patient. A non-adhesive dead zone may be formed on a periphery of the anchor that will not attach to the patient's skin and that will assist the surgeon in the removal of the device upon completion of the procedure. The materials and dimensions of the anchor are selected to allow the device to releasably conform to the contours of the patient's skin.

### PRIORITY DATA

This application claims priority to and the benefit of the filing date of U.S. Provisional Application No. 63/497,399, filed Apr. 20, 2023, titled “Abdominal Tissue Lifting Device,” and U.S. Provisional Application No. 63/503,060, filed May 18, 2023, titled “Abdominal Tissue Lifting Device,” each of which is incorporated herein by reference in their entireties.

### FIELD OF THE INVENTION

The subject invention relates generally to the field of patient tissue lifting devices for use in surgery, and more particularly, to an abdominal tissue lifting device for elevating tissue of a patient in laparoscopic surgery.

### BACKGROUND OF THE INVENTION

Laparoscopic surgery has been available for many years and has become more widely used due to technological advancements in surgical instruments and video imaging systems. As part of the laparoscopic procedure, various instruments such as a Veress needle, a trocar or access ports are typically inserted through tissue of the abdominal wall for access to the abdominal cavity. Carbon dioxide may then be used to insufflate the abdominal cavity to thereby form a pneumoperitoneum or space within the abdominal cavity to operate. Prior to insufflation, it is desirable to separate the abdominal wall and muscle from the organs below so as to not only assist in the creation of an operating space but to also help the surgeon in the safe placement of the laparoscopic instruments to reduce the potential for injury to the patient.

Various techniques and instruments have been developed over the years to assist in the elevation or lifting of the abdominal wall for instrument placement during laparoscopic surgery. One example is described in U.S. Pat. No. 5,415,160, entitled “Surgical Lift Method and Apparatus”, issued to Ortiz et al. on May 16, 1995 (“the '160 Patent”). The '160 Patent describes a surgical lift device that includes a gripping portion for adhesively contacting and holding an external skin surface adjacent the abdominal wall. The surgical lift device is used in conjunction with a support structure that is designed to extend across the center portion of the operating table and apply an upward force by a chain to the surgical lift device to elevate and hold the abdominal wall to a desired position.

Another example of an abdominal wall elevation device is shown in U.S. Pat. No. 10,363,380, entitled “Device and Methods for Lifting Patient Tissue During Laparoscopic Surgery”, issued to Taylor et al. on Jul. 30, 2019 (“the '380 Patent”). The '380 Patent discloses a surgical device that provides a suction force against the patient's body to lift tissue for insertion of a trocar or Veress needle.

A further example is described in U.S. Pat. No. 10,722,227, entitled “Medical Device for Use in the Creation of a Temporary Pneumoperitoneum”, issued to Foley on Jul. 28, 2020 (“the '227 Patent”). The '227 Patent utilizes a dome shaped device and negative pressure generated from external operating room suction to raise the abdominal wall of the patient within the dome, and thereby create an expanded abdominal cavity.

While these known abdominal wall elevation devices may provide certain benefits, many laparoscopic surgeries are still presently performed with the abdominal wall being lifted either by manual grasping or by towel clips attached to the patient's skin that serve as a handle to elevate the abdominal tissue. Accordingly, there is clearly a need for an improved abdominal tissue lifting device that is relatively simple, cost-effective and easy to use during laparoscopic surgery.

### SUMMARY OF THE INVENTION

It is an object of the invention to provide an improved tissue lifting device for elevating tissue of a patient in a medical procedure.

It is another object of the invention to provide an improved hand held abdominal tissue lifting device that is adhesively attachable to skin of a patient for lifting during laparoscopic surgery.

### DESCRIPTION OF THE FIGURES

FIG. 1 is an image of the abdominal tissue lifting device of the subject invention shown in use during laparoscopic surgery.

FIG. 2 is a top perspective view of the abdominal tissue lifting device shown in FIG. 1.

FIG. 3 is a bottom perspective view of the base of the abdominal tissue lifting device of FIG. 2.

FIG. 4 is a bottom plan view of the base of FIG. 3.

FIG. 5 is an exploded side view of the base of FIG. 4.

FIG. 6 is a bottom plan view of the anchor plate of the abdominal tissue lifting device of FIG. 2.

FIG. 7 is an exploded side view of the anchor plate of FIG. 6.

FIG. 8 is a top perspective view of the handle of the abdominal tissue lifting device of FIG. 2.

FIG. 9 is a top perspective view of the strap used in the formation of the handle of FIG. 8 shown in a condition prior to handle formation.

FIG. 10 is a top plan view of the strap shown in FIG. 9.

FIG. 11 is an exploded side view of the strap of FIG. 10.

FIG. 12 is an image of the abdominal tissue lifting device of the subject invention shown with the handle of FIG. 8 being in a folded, collapsed condition.

FIG. 13 is an exploded top perspective view of an alternative embodiment of an abdominal tissue lifting device of the subject invention.

FIG. 14 is a top perspective view of the strap used in the formation of the handle of FIG. 13 shown in a condition prior to handle formation.

FIG. 15 is a bottom plan view of the strap shown in FIG. 14.

FIG. 16 a top perspective view of a further alternative embodiment of an abdominal tissue lifting device of the subject invention.

FIG. 17 is a top plan view of the strap of the abdominal tissue lifting device of FIG. 16 prior to formation into the abdominal tissue lifting device.

FIG. 18 is an exploded side view of the strap of FIG. 17.

FIG. 19 is a top perspective view of yet a further alternative embodiment of an abdominal tissue lifting device of the subject invention.

FIG. 20 is a top plan view of the sheet of the of the abdominal tissue lifting device of FIG. 19 prior to formation into the abdominal tissue lifting device.

FIG. 21 is an exploded side view of the strap of FIG. 20.

FIG. 22 is a top perspective view of still a further alternative embodiment of an abdominal tissue lifting device of the subject invention comprising a handle and an anchor.

FIG. 23 is a side elevation view of the handle of the abdominal tissue lifting device of FIG. 22.

FIG. 24 is a bottom plan view of a base for forming the anchor of the abdominal tissue lifting device of FIG. 22 prior to formation.

FIG. 25 is an exploded side view of the components of the base of FIG. 24.

FIG. 26 is a bottom plan view of the handle of FIG. 23 positioned over the base of FIG. 24 prior to folding into the abdominal tissue lifting device of FIG. 22.

FIG. 27 is a top perspective view of the abdominal tissue lifting device after formation with the handle rotated about 180 degrees away from the anchor portions of the device.

FIG. 28 is a side elevation view of the abdominal tissue lifting device of FIG. 22 with the anchor portions lying in a common plane and the handle in an upright position.

FIG. 29 is a top perspective view of the abdominal tissue lifting device with the handle rotated parallel to the anchor portions and contained within a shipping tray.

FIG. 30 is a side elevation view of the an alternative handle of the abdominal tissue lifting device.

### DESCRIPTION OF THE EMBODIMENTS

For the purposes of promoting and understanding of the principles of the invention, reference will now be made to the embodiments illustrated in the drawings and described in the following written specification. It is understood that no limitation to the scope of the invention is thereby intended. It is further understood that the present invention includes any alterations and modifications to the illustrated embodiments and includes further applications of the principles of the invention as would normally occur to one skilled in the art to which this invention pertains.

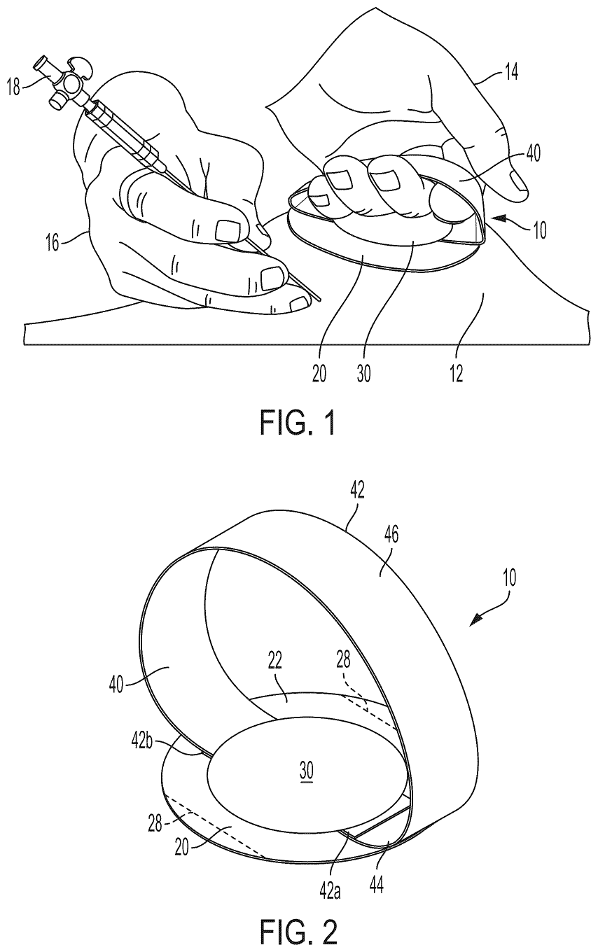

Referring to FIG. 1, a hand-held abdominal tissue lifting device 10 is shown in use during laparoscopic surgery. Lifting device 10 is adhesively secured to external tissue 12 of the patient's abdomen, as will be more fully described. As shown, the surgeon is able to grasp lifting device 10 with one hand 14 and pull abdominal tissue 12 upwardly from the patient to create an abdominal cavity for the surgeon to operate. The other hand 16 of the surgeon is free to use an instrument, such as a Veress needle 18 for insertion into the expanded abdominal cavity whereby carbon dioxide or other suitable gas may be introduced through the Veress needle 18 to sufflate the abdominal cavity. Other instruments, such as trocars or surgical cannulas may also be introduced into the expanded abdominal cavity during elevation of the abdominal wall provided by the pulling action of the abdominal tissue lifting device 10 by the surgeon.

With reference to FIG. 2, a description of the components of abdominal tissue lifting device 10 is provided. Lifting device 10 comprises a base 20, an anchoring plate 30 and a handle 40. As shown, a portion of handle 40 is attached in lifting device 10 between a lower surface of anchor plate 30 and an upper surface of base 20, as will be described.

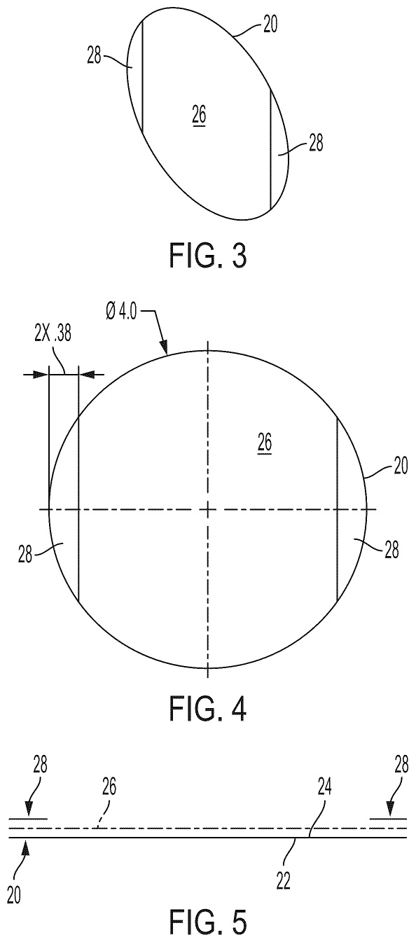

Turning now to FIGS. 3-5, details of base 20 are now described. Base 20 in the particular arrangement shown is generally circular, although other configurations such as oval, rectangular or square may also be used. Base 20 includes an upper surface 22 and a lower surface 24. Lower surface 24 is substantially entirely covered with a layer of adhesive 26 that provides a single anchor point for attachment to the skin of a patient. A pair of thin films 28 is attached to adhesive layer 26 at opposing diametric ends of base 20 to form chordal dead zones that will not attach to the patient's skin and that will assist the surgeon in the removal of tissue lifting device 10 upon completion of the procedure, as will be explained. Each film 28 may be formed of polyester or other suitable material with a thickness in the range of 0.001 to 0.125 inches.

Base 20 may be formed of a non-woven polyethylene, although other suitable materials such as polyester may also be used. In one particular arrangement base 20 is formed to have a diameter of approximately 4.0 inches resulting in a tissue contact area of approximately 12.6 square inches. Such an area is desirable as it provides a size and configuration that would not substantially cover the abdomen but would potentially allow the use of only a single lifting device 10 instead of two or more during surgery while permitting other port entry sites. It should be understood that a base 20 having an area in the range of 1.0 to 35.0 square inches may also be used. In a particular arrangement, base 20 may have a thickness in the range of 0.001 to 0.250 inches.

Adhesive 26 in certain instances is selected to be capable of lifting a load of at least 10 pounds and to hold such load for a period of at least 30 seconds. Otherwise, adhesive 26 is selected to be sufficiently capable of tenting the abdomen for insertion by a Veress needle, trocar or other instruments. As such, adhesive 26 may be comprised of biocompatible materials including synthetic rubber, silicone, or acrylic based adhesives, or other suitable commercially available adhesive.

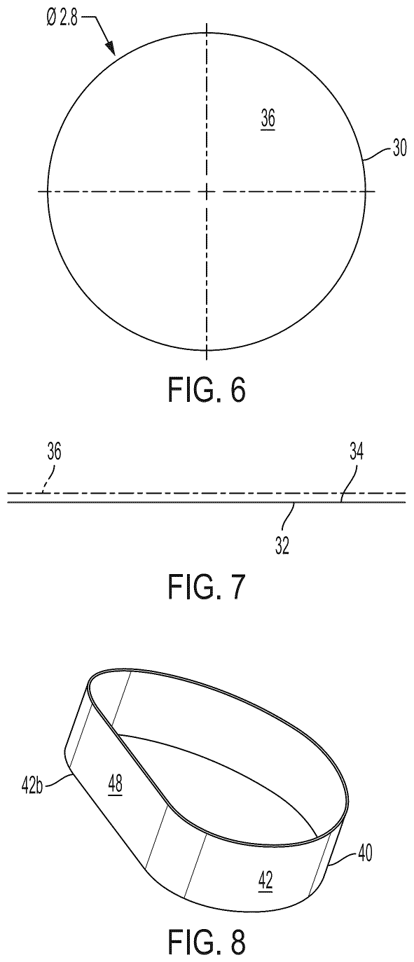

Referring now to FIGS. 6-7, details of anchor plate 30 are now described. Anchor plate 30 in the particular arrangement shown is generally circular, although other configurations compatible with but smaller than the configuration of base 20 may be used. Anchor plate 30 includes an upper surface 32 and a lower surface 34. Lower surface 34 is substantially entirely covered with a layer of adhesive 36. Anchor plate 30 may also be formed of a non-woven polyethylene, although other suitable materials such as polyester may be used. In one particular arrangement anchor plate 30 is formed to have a diameter of approximately 2.8 inches when the diameter of base 20 is approximately 4.0 inches. It is desirable that the diameter of anchor plate 30 be smaller than the diameter of base 20 to decrease the propensity of base 20 peeling along the circumferential edge, but large enough to transfer lifting load radially from the center of base 20 upon the application of a substantially orthogonal lifting force by handle 40. Such a ratio of the diameter of anchor plate 30 to the diameter of base 20 is preferably about 70%, although such ratio may also be effective in the range of 50%-90%. In a particular arrangement anchor plate 30 may have a thickness in the range of 0.001 to 0.125 inches.

Adhesive 36 is selected to have a stronger adhesive strength than the adhesive strength of adhesive 26 of base 20. Such stronger adhesive strength is desirable to prevent anchor plate 30 and hence handle 40 from detaching from base 20 before base 20 detaches from the skin of the patient. As such, adhesive 36 may be comprised of biocompatible materials including polyethylene, polyurethane, acrylic, epoxy, polyimide, urethane, anaerobic, or ultraviolet light adhesives. The combination of materials and dimensions of base 20 and plate 30 are selected to allow device 10 to be sufficiently rigid so as to be self-sustaining while being sufficiently supple so as to conform to the contours of the patient's tissue.

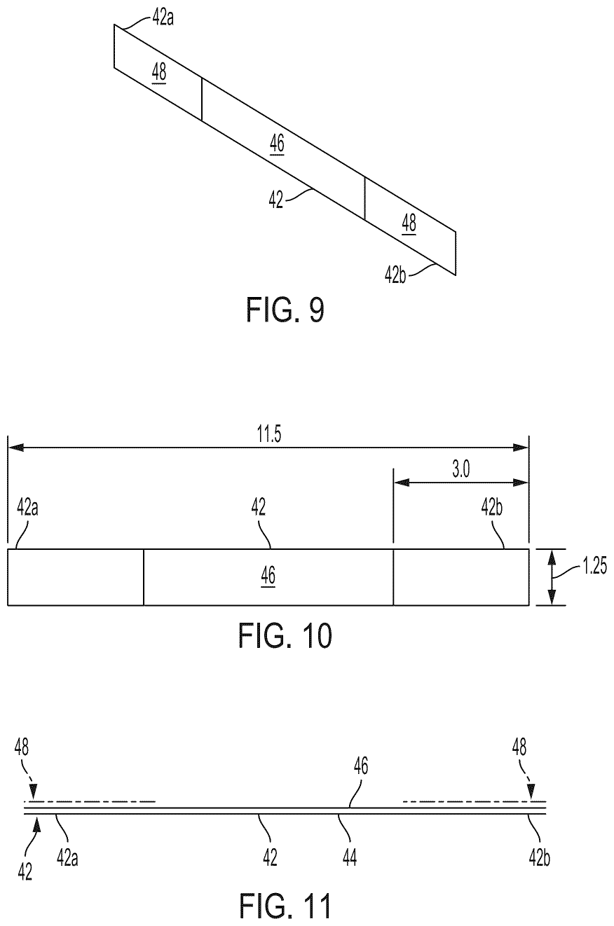

Turning now to FIGS. 8-11, further details of handle 40 are described. Handle 40 is formed from a thin, elongate strap 42 that is bent into a loop configuration as shown in FIG. 8. Strap 42 in this form is substantially flat having a bottom surface 44, a top surface 46 and first and second opposing ends 42a and 42b. A layer of adhesive is formed respectively on top surface 46 at each end 42a and 44b, with no adhesive there between.

Strap 42 may be formed of a polyethylene, although other suitable materials may also be used. For example, strap 42 may comprise a biaxially oriented laminate of biocompatible polyesters, polypropylenes, synthetics, spun bond nonwovens or melt-blown nonwoven materials to prevent or reduce unacceptable elongation of strap 42 when immobility is desired. In the arrangement of lifting device 10 described above wherein base 20 has a diameter of 4.0 inches, strap 42 may be configured to have a length of 11.5 inches, a width of 1.25 inches and a thickness of approximately 0.002 inches. It should be understood that depending upon the size and configuration of base 20, strap 42 may be formed to have other dimensions.

Handle 40 is formed by bending strap 42 into a loop such that the layer of adhesive 48 at strap end 42a is placed adjacent to and above bottom surface 44 of strap 42 at strap end 42b. The adhesive 48 at strap end 42a joins opposing ends strap ends 42a and 42b, as shown in FIG. 8, with the layer of adhesive 48 at strap end 42b being exteriorly exposed at the bottom of handle 40. Suitable removable liners (not shown) may cover the adhesives 48 at both ends 42a and 42b until assembly with base 20 and anchor plate 30. As attached, handle 40 resides within the perimeter of base 20.

Having described the components of abdominal tissue lifting device 10, the assembly of device 10 is described with further reference to FIG. 2. Handle 40 is attached to upper surface 22 of base 20 by the layer of adhesive 48 disposed at the bottom of strap end 42b of handle 40. Handle 40 is oriented on upper surface 22 of base 20 such that the length of strap 42 is generally parallel to and substantially centrally between films 28 on base 20. Anchor plate 30 is then applied over strap 42 of handle 40 at the approximate center of base 20, whereat anchor plate 30 is joined to handle 40 and base 20 by adhesive 36 on the bottom surface 34 of anchor plate 30. As such, the adhesively joined ends 42a and 42b of handle strap 42 are sandwiched between anchor plate 30 and base 20, decreasing the potential of handle 40 delaminating from base 20 during use. It should be appreciated that other techniques may also be considered for attaching anchor plate 30 to base 20 that would eliminate or diminish the potential of handle 40 separating from base 20 during the elevation process. For example, anchor plate 30 may be sewed or riveted to base 20, or attached by ultrasonic welding. In addition, handle 40 may also be sewed, riveted or ultrasonically welded to anchor plate 30 prior to assembly to base 20. Further, lower surface 34 of anchor plate 30 and upper surface 22 of base 20 may be configured to have cooperative hook and loop features for fastening anchor plate 30 and base 20 together with handle 40. In such a hook and loop arrangement, the strength of the hook and loop fastener must be greater than the adhesive strength provided by adhesive 26 that attaches base 20 to the skin of the patient. Yet further, handle 40 may be sewed, riveted or ultrasonically welded directly to base 20, or handle 40 and base 20 may be formed integrally as a one-piece device.

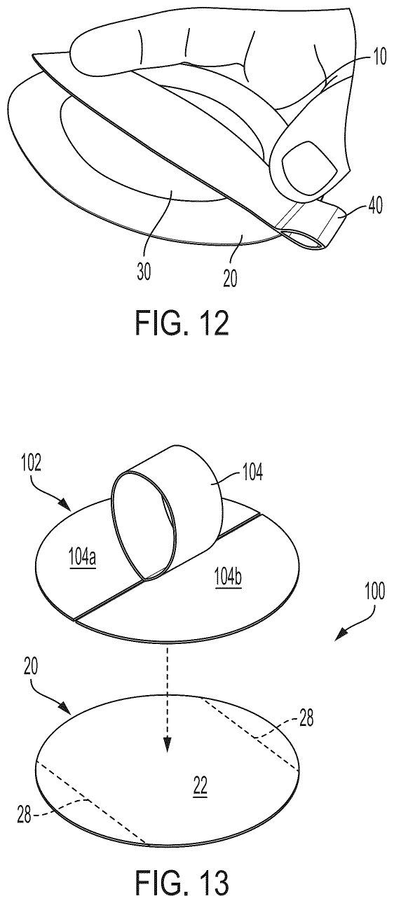

The materials of lifting device 10 described herein allow lifting device 10 to be sterilized prior to or with associated packaging, such as by gamma radiation or ethylene oxide. In addition, the materials of device 10 may be selected to render device 10 radiolucent such that suitable imaging may occur while device 10 is attached to a patient. Further, the material and thickness of handle 40 allow handle 40 to collapse and fold into a substantially flat condition as shown in FIG. 12, which condition facilitates packaging. Prior to packaging, a one-piece liner (not shown) may be removably attached to adhesive 26 on lower surface 24 of base 20. A removal tab may be provided on the one-piece liner to allow the surgeon to grasp and remove the liner in one piece for discarding. Alternatively, a two-piece split liner may be removably attached to adhesive 26. This would allow the surgeon to place lifting device 10 in a desired location on the patient's skin, then remove the liner from the medial to the distal direction one half at a time. A further alternative is to form a removable liner as part of the packaging, such that upon removing lifting device 10 from the packaging, lifting device 10 also will be separated from the liner which remains with the packaging for discarding.

In use, reference is again made to FIG. 1. Upon determining a desirable location on the patient's abdomen, adhesive 26 on lower surface 24 of base 20 is appropriately attached to the patient's skin. The folded collapsed handle 40 is sufficiently sized and expandable to allow the surgeon to slide his/her fingers of one hand into the handle 40 for grasping. Manual upward force is applied to base 20 through handle 40 with the load being transferred radially through anchor plate 30 from the center of device 10. Since anchor plate 30 does not extend to the periphery of base 20, the peel propensity at the perimetric edge of base 20 is reduced. In addition, the attached anchor plate 30 further decreases the potential of delamination of handle 40 from base 20 during the application of the upward pulling force by the surgeon. As the abdominal wall is elevated upon the application of the upward force applied to lifting device 10, the surgeon may then use a Veress needle 18, a trocar or other instruments to gain access into the expanded abdominal cavity for surgery. Upon completion of use, lifting device 10 may be removed from the skin of the patient by the surgeon slipping his/her fingers beneath one of the films 28 on the diametrically opposed edges of device 10, which are not adhered to the patient, so as to grab base 20 and peel device 10 from the patient. Inasmuch as lifting device 10 is intended as a disposable device for single use, upon removal lifting device 10 may be suitably discarded.

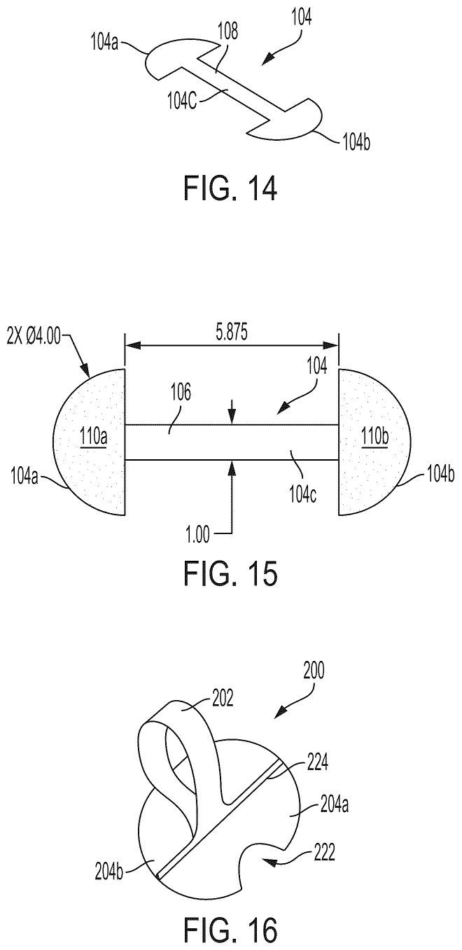

With reference now to FIGS. 13-15, an alternative embodiment of a hand held abdominal tissue lifting device 100 is described. Lifting device 100 comprises two components, namely base 20 and a handle 102. Base 20 is essentially the same as base 20 described hereinabove with respect to lifting device 10. Handle 102 is formed from a thin, elongate strap 104 that is bent into a loop configuration as shown in FIG. 13 so as to allow for the insertion of a practioner's fingers. Prior to forming the loop configuration, strap 104 is substantially flat as illustrated in FIGS. 14 and 15, having a bottom surface 106, a top surface 108, first and second opposing anchor portions 104a and 104b, and a connecting portion 104c that joins anchor portions 104a and 104b. Anchor portions 104a and 104b are each shaped in the form of a semicircle. In one arrangement, each anchor portion 104a and 104b may have the same diameter as the diameter of base 20. In other arrangements, the diameter of each anchor portion 104a and 104b may be less than the diameter of base 20 and similar to the diameter of anchor plate 30. Connecting portion 104c has a width dimension that is less than the diameters of anchor portions 104a and 104b,

A layer of adhesive 110a and 110b is formed respectively on the bottom surface 106 of each anchor portion 104a and 104b. Each adhesive layer 110a and 110b is in one arrangement the same as adhesive layer 36 that is applied to the lower surface 34 of anchor plate 30 described above. As such, adhesive layers 110a and 110b are selected to have a stronger adhesive strength than the adhesive strength of adhesive 26 of base 20. No adhesive is applied to the bottom surface 106 of connecting portion 104c.

Strap 104 that forms handle 102 may be polyethylene, although other suitable materials may also be used. For example, strap 104 may comprise a biaxially oriented laminate of biocompatible polyesters, polypropylenes, synthetics, spun bond nonwovens or melt-blown nonwoven materials to prevent or reduce unacceptable elongation of strap 104 when immobility is desired. In an arrangement of lifting device 100 wherein base 20 has a diameter of 4.0 inches, the semicircular diameters of anchor portions 104a and 104b may also be 4.0 inches. In such arrangement, connecting portion may have a width of 1.00 inches, a length of 5.875 inches between the anchor portions 104a and 104b, and a thickness of approximately 0.002 inches. It should be understood that depending upon the size and configuration of base 20, strap 104 may be formed to have other dimensions, and that anchor portions 104a and 104b may have shapes other that semicircles.

Handle 102 is formed by bringing anchor portions 104a and 104b together medially to form a loop and then adhering portions 104a and 104b to upper surface 22 of base 20. Suitable removable liners (not shown) may cover the adhesives 110a and 110b on anchor portions 104a and 104b until assembly with base 20. As attached, handle 102 resides within the perimeter of base 20. Lifting device 102 may then be used in the same manner as lifting device 10 described above.

It should be understood that other techniques may also be considered for forming tissue lifting device 100. For example, anchor portions 104a and 104b may be sewed or ultrasonically welded to base 20 instead of being attached adhesively. Also, instead of adhesive layers 110 and 110b being applied to anchor portions 104a and 104b, an adhesive layer comparable to adhesives 110a and 110b may be applied to upper surface 22 of base 20 to adhesively attach anchor portions 104a and 104b to base 20. In addition, handle 102 and base 20 may be formed as an integral one-piece device. Lastly, in some situations handle 102 may be used without base 20 for direct attachment to skin of a patient. In such instance, adhesive layers 110a and 110b may comprise adhesives that are at least comparable in strength to adhesive layer 26 that is included on the lower surface 24 of base 20. In this arrangement, there would be two anchor points that provide spaced attachment to the skin of the patient while connecting portion 104c provides an unattached loop for receipt of the practitioner's fingers.

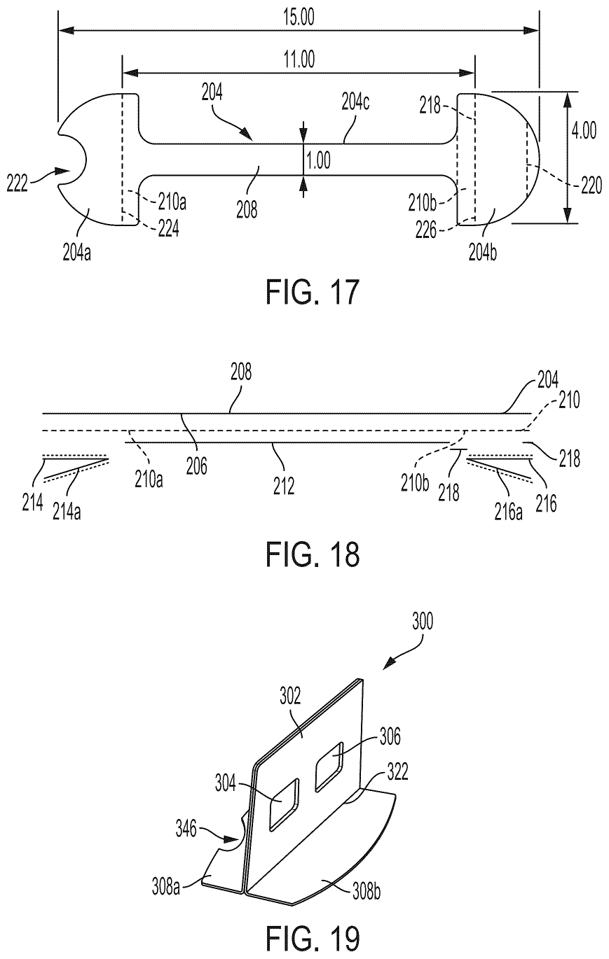

With reference now to FIGS. 16-18, a further alternative embodiment of a hand held abdominal tissue lifting device 200 is described. Lifting device 200 has similarities to lifting device 100, but unlike lifting device 100, which comprises two components, namely base 20 and a handle 102, lifting device 200 does not require a separate base for attachment to a patient. Lifting device 200, as illustrated in FIG. 15, comprises a handle 202 and a pair of opposed anchor portions 204a and 204b. Anchor portions 204a and 204b are directly adhesively attached to skin of the patient, as will be described. Handle 202 is formed from a thin, elongate strap 204 that is bent into a loop configuration as shown in FIG. 16 to allow for the insertion of a practitioner's fingers. Prior to forming the loop configuration, strap 204 is a substantially flat, integral one-piece component, as illustrated in FIGS. 17 and 18. Strap 204 has a bottom surface 206, a top surface 208, first and second opposing anchor portions 204a and 204b, and a connecting portion 204c that joins anchor portions 204a and 204b. In one arrangement, anchor portions 204a and 204b are each shaped in the form of a semicircle. In this arrangement, each anchor portion 204a and 204b may have the same diameter as the diameter of base 20, described above with respect to lifting device 10. In other arrangements, the diameter of each anchor portion 204a and 204b may be greater than or less than the diameter of base 20. Connecting portion 204c has a width dimension that is less than the diameters of anchor portions 204a and 204b.

Strap 204 that forms handle 202 may be polyethylene, although other suitable materials may also be used. For example, strap 204 may comprise a biaxially oriented laminate of biocompatible polyesters, polypropylenes (BOPP), synthetics, spun bond nonwovens or melt-blown nonwoven materials to prevent or reduce unacceptable elongation of handle 202 when immobility is desired. In an arrangement of lifting device 200 the semicircular diameters of anchor portions 204a and 204b may be 4.0 inches. In such arrangement, connecting portion 204c may have a width of 1.00 inches, and strap 204 may have an extent of 11.0 inches between center points of anchor portions 204a and 204b, an overall length of 15.0 inches, and a thickness of approximately 0.003 to 0.100 inches. It should be understood that strap 204 may be formed to have other dimensions and that anchor portions 204a and 204b may have shapes other that semicircles.

A layer of adhesive 210 is included on the bottom surface 206 of strap 204. In the arrangement shown, adhesive 210 extends along the entire length of strap 204, i.e., on the bottom surfaces 206 of opposing anchor portions 204a and 204b, as well as on the bottom surface 206 of connecting portion 204c. In some instances, adhesive 210 may be included on only the bottom surfaces 206 of opposing anchor portions 204a and 204b. Adhesive layer 210, in one arrangement, is the same as adhesive layer 26 that is applied to the lower surface 24 of base 20 described above. In other instances, the strength of adhesive 210 is preferably stronger than that of adhesive layer 26, and may comprise high initial tact, extended wear, acrylic adhesive.

In the arrangement where adhesive layer 210 is included on the bottom surface 206 of connecting portion 204c, adhesive layer 210 along the length of connecting portion 204c may be rendered non-adhesive by covering adhesive layer 210 with an adhesive deadener 212. Bottom surface 206 of connecting portion 204c forms the underside of the loop portion of deice 200 which would be grasped by a practitioner. As such, it is preferable that this surface be non-tacky. Adhesive deadener 212 may be a non-adhesive film or a spray or coating applied to adhesive layer 210. Adhesive deadener 212 extends substantially the length of connecting portion 204c without covering the bottom surface 206 of anchor portions 204a and 204b.

A shown in FIG. 18, a removable liner 214 may cover adhesive layer 210 on the bottom surface 206 of anchor portion 204a. Liner 214 may have a tab 214a to facilitate removal from adhesive 210 for assembly, as will be described. Liner 214 is configured to cover all of adhesive 210 on the bottom surface 206 of anchor portion 204a except for an exposed strip 210a of adhesive that extends laterally across anchor portion 204a adjacent connecting portion 204c, as shown in FIGS. 17 and 18. Strip 210a of adhesive lies between liner 214 and one axial end of adhesive deadener 212.

At the opposite end of strap 204, a removable liner 216 may cover adhesive layer 210 on the bottom surface 206 of anchor portion 204b. Liner 216 may have a tab 216a to facilitate removal from adhesive 210 for assembly, as will be described. Liner 216 is configured to cover all of adhesive 210 on the bottom surface 206 of anchor portion 204b except for an exposed strip 210b of adhesive 210 that extends laterally across anchor portion 204b adjacent connecting portion 204c, as shown in FIGS. 17 and 18. Strip 210b of adhesive lies between liner 216 and the other axial end of adhesive deadener 212. A rigidifier 218 is attached adhesively to anchor portion 204b on exposed adhesive strip 210b, with rigidifier 218 extending laterally across substantially the width of anchor portion 210b. A layer of adhesive may be applied to rigidifier 218 prior to attachment to strap 204 to further enhance the rigidity of rigidifier 218 and the securement to strap 204. Rigidifier 218 may be approximately 0.003 to 0.100 inches thick and be comprised of polyethylene or other suitable material. The function and purpose of rigidifier 218 are described below.

A thin film 220 similar to film 28 described above may be attached to adhesive layer 210 at one or both ends of strap 204 to form chordal dead zones on anchor portion 204a and/or 204b that will not attach to the patient's skin and that will assist the surgeon in the removal of tissue lifting device 200 upon completion of the procedure, as will be explained. Each film 220 may be formed of polyester or other suitable material with a thickness in the range of 0.001 to 0.125 inches. A cutout 222 may be formed into the periphery of one of the anchor portions 204a or 204b to keep an area of the patient's skin, such as the navel area, open upon attachment of device 200 to the patient.

Lifting device 200 is formed by bringing anchor portions 204a and 204b together medially whereby adhesive strip 210a is adhesive attached to rigidifier 218. As formed, anchor portions 204a and 204b face each other. Connecting portion 204c may be temporarily flattened such that the entire device 200 is substantially flat. Device 200 may be packaged in this condition.

In use, anchor portions 204 a and 204b may be folded away from each other along fold lines 224 and 226 to project outwardly transversely from handle 202, as depicted in FIG. 15. Handle 202 may be formed into a loop as also shown in FIG. 15. Device 200 may be attached to a selected area of the patient's skin by removing one of liners 214 or 216 to expose adhesive layer 210 once a liner is removed. For example, if liner 214 is removed first, anchor portion 204a is then adhesively attached to the skin of the patient. Upon attachment of anchor portion 204a, liner 216 is then removed and anchor portion 204b is attached to the patient's skin completing the process of attaching device 200 to the skin of the patient. If the attachment is near the patient's navel, the cutout portion 222 may be aligned with the navel such that the device 200 will not be adhered to the patient's navel.

Handle 202 is sufficiently sized to allow the surgeon to slide his/her fingers of one hand into the loop of handle 202 for grasping. Manual upward force is applied to anchor portions 204a and 204b with the load being transferred radially outwardly through rigidifier 218 from the center of device 200. This load transfer allows handle 202 to be pulled outwardly from the patient and provide a sufficient force to lift the patient's skin without the handle collapsing inwardly during the pulling process. The surgeon may then use a Veress needle 18, a trocar or other instruments to gain access into the expanded abdominal cavity for surgery. Upon completion of use, lifting device 200 may be removed from the skin of the patient by the surgeon slipping his/her fingers beneath one of the films 220 on the diametrically opposed edges of device 200, which are not adhered to the patient, so as to grab and peel device 200 from the patient. Inasmuch as lifting device 200 is intended as a disposable device for single use, upon removal lifting device 200 may be suitably discarded.

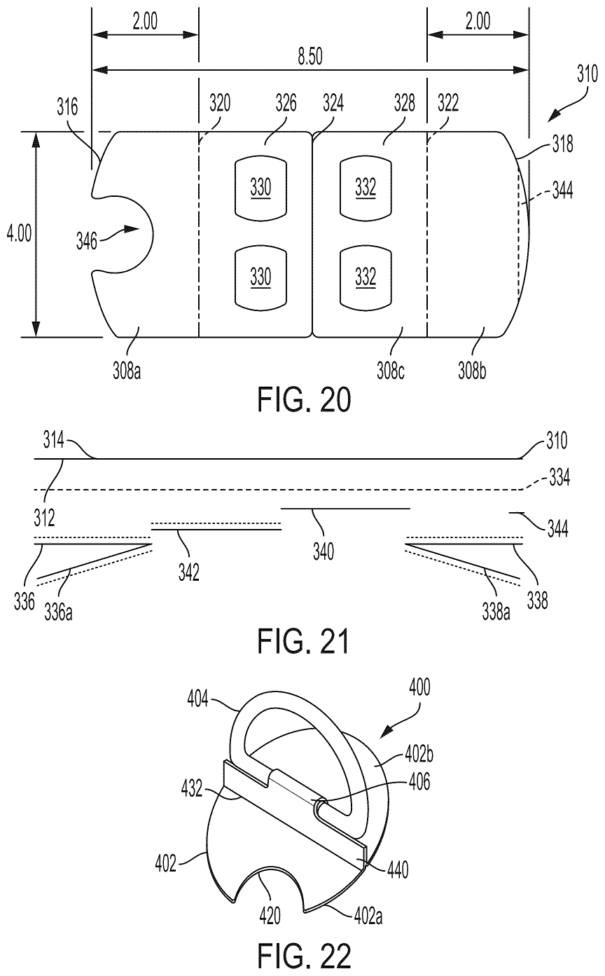

Turning now to FIGS. 19-21, yet a further alternative embodiment of an abdominal hand held tissue lifting device 300 is described. Lifting device 300 has similarities to lifting device 200, but unlike lifting device 200, which comprises a looped handle 202, lifting device 300 comprises a generally rectangular handle 302 that has finger openings 304 and 306, and a pair of opposed anchor portions 308a and 308b projecting transversely form handle 302. Like lifting device 200, anchor portions 308a and 308b are directly adhesively attached to skin of the patient. Handle 302 is formed from a thin, flat sheet 310 that is folded into the configuration as shown in FIG. 19 to allow for the insertion of a practitioner's fingers into openings 304 and 306. Prior to forming the device 300, sheet 310 is a substantially flat, integral one-piece component, as illustrated in FIGS. 20 and 21. Sheet 310 has a bottom surface 312, a top surface 314, first and second opposing anchor portions 308a and 308b, and a connecting portion 308c that joins anchor portions 308a and 308b. Anchor portion 308a may have a curved edge 316 and anchor portion 308b may have a curved edge 318 at the opposite end of sheet 310.

As shown in FIG. 20 sheet 310 defines three fold lines, 320, 322 and 324 that extend laterally across the width of sheet 310 about which sheet 310 is folded to form lifting device 300. Fold line 320 defines a boundary between anchor portion 308a and one end of connecting portion 308c and fold line 322 defines a boundary between anchor portion 308b and an opposite end of connecting portion 308c. Fold line 324 is disposed substantially centrally on sheet 310 such that upon sheet 310 being folded connecting portion 308c is divided into two halves 326 and 328. A first pair of windows 330 is formed through connecting portion half 326 and a second pair of windows 332 is formed through connecting portion half 328. Windows 330 and 332 are sized for receipt of a surgeon's fingers. Windows 330 and 332 are oriented such that upon sheet 310 being folded along fold line 324 into device 300, windows 330 and 332 will align to essentially form a single pair of windows 304 and 306, as shown in FIG. 19, that serve as openings for a surgeon to grasp during use, as will be described.

A shown in FIG. 21, a layer of adhesive 334 is included on the bottom surface 312 of sheet 310. In the arrangement shown, adhesive 334 extends along the entire length of sheet 310, i.e., on the bottom surfaces 312 of opposing anchor portions 308a and 308b, as well as on the bottom surface 312 of connecting portion 308c. Adhesive layer 334 is the same as adhesive layer 210 described above with respect to lifting device 200. A removable liner 336 may cover adhesive layer 334 on the bottom surface 312 of anchor portion 308a. Liner 336 may have a tab 336a to facilitate removal from adhesive 334 for assembly, as will be described.

At the opposite end of sheet 310, a removable liner 338 may cover adhesive layer 334 on the bottom surface 312 of anchor portion 308b. Liner 338 may have a tab 338a to facilitate removal from adhesive 334 for assembly, as will be described. A rigidifier 340 is attached adhesively to connecting portion half 328 on adhesive layer 334. Rigidifier 340 extends laterally across substantially the width of sheet 310. Rigidifier 336 may be approximately 0.003 to 0.100 inches thick and be comprised of polyethylene or other suitable material. The function and purpose of rigidifier 336 is essentially the same as the rigidifier 218 of lifting device 200. Another removable liner 342 may cover adhesive layer 334 on the bottom surface 312 of connecting half 326.

A thin film 344 similar to film 220 described above may be attached to adhesive layer 334 at one or both ends of anchor portions 308a and 308b to form chordal dead zones that will not attach to the patient's skin and that will assist the surgeon in the removal of tissue lifting device 300 upon completion of the procedure, as will be explained. Each film 342 may be formed of polyester or other suitable material with a thickness in the range of 0.001 to 0.125 inches. A cutout 346 similar to cutout 222 of device 200 may be formed into the periphery of one of the anchor portions 308a or 308b to keep an area of the patient's skin, such as the navel area, open upon attachment of device 300 to the patient.

Sheet 310 may be polyethylene, although other suitable materials may also be used. For example, strap 104 may comprise a biaxially oriented laminate of biocompatible polyesters, polypropylenes (BOPP), synthetics, spun bond nonwovens or melt-blown nonwoven materials to prevent or reduce unacceptable elongation of handle 302 when immobility is desired. In an arrangement of lifting device 300, sheet 310 has a width of 4.00 inches, a length of 8.50 inches and a thickness of approximately 0.003 to 0.100 inches. In such arrangement, anchor portions 308a and 308b may each have a length of 2.00 inches. It should be understood that sheet 310 may be formed to have other dimensions and that anchor portions 308aa and 308b may have shapes other than curved edges 316 and 318, respectively.

Handle 302 is formed by folding sheet 310 about fold line 324 such that connecting portion halves 326 and 328 face each other with windows 330 in alignment with windows 332. Upon removing liner 342, adhesive layer 334 on bottom surface 312 of connecting portion half 326 is adhered to rigidifier 340. As formed, anchor portions device 300 is substantially flat with anchor portions 308a and 308b facing each other. Device 300 may be may be folded and packaged with handle of 302 substantially flat against anchor portions 308a or 308b.

In use, anchor portions 308a and 308b may be folded about respective fold lines 320 and 322 such that anchor portions 308a and 308b project outwardly transversely from handle 202, as depicted in FIG. 19. Device 300 may be attached to a selected area of the patient's skin by removing one of liners 336 or 338 to expose adhesive layer 334 once a liner is removed. For example, if liner 336 is removed first, anchor portion 308a is then adhesively attached to the skin of the patient. Upon attachment of anchor portion 308a, liner 338 is then removed and anchor portion 308b is attached to the patient's skin completing the process of attaching device 300 to the skin of the patient. In the event that the attachment is near the patient's navel, cutout 346 in an anchor portion 308a or 30b may be aligned such that the device 300 will not be adhered to the patient's navel.

Handle 302 is sufficiently sized to allow the surgeon to slide his/her fingers of one hand into the openings 304 and 306 for grasping. Manual upward force is applied to anchor portions 308a and 304b with the load being transferred laterally outwardly through rigidifier 340 from the center of device 300. This load transfer allows handle 302 to be pulled outwardly from the patient and provide a sufficient force to lift the patient's skin without the handle collapsing inwardly during the pulling process. The surgeon may then use a Veress needle 18, a trocar or other instruments to gain access into the expanded abdominal cavity for surgery. Upon completion of use, lifting device 300 may be removed from the skin of the patient by the surgeon slipping his/her fingers beneath one of the films 344 on the axially opposed edges of device 300, which are not adhered to the patient, so as to grab and peel device 300 from the patient. Inasmuch as lifting device 300 is intended as a disposable device for single use, upon removal lifting device 300 may be suitably discarded.

With reference now to FIGS. 22-30, still a further alternative embodiment of a hand held abdominal tissue lifting device 400 is described. Lifting device 400, as shown in FIG. 22 has similarities to lifting device 200, but unlike lifting device 200, lifting device 400 comprises a flexible anchor 402 and a separate handle 404 that are assembled together to form lifting device 400, as will be described. Anchor 402 comprises a pair of opposed anchor portions 402a and 402b that are each configured to be directly adhesively attached to skin of the patient, as will also be described. Handle 404 is supported by and rotatable relative to anchor 402 within a hinge 406 formed between anchor portions 402a and 402b, as will be detailed hereinbelow.

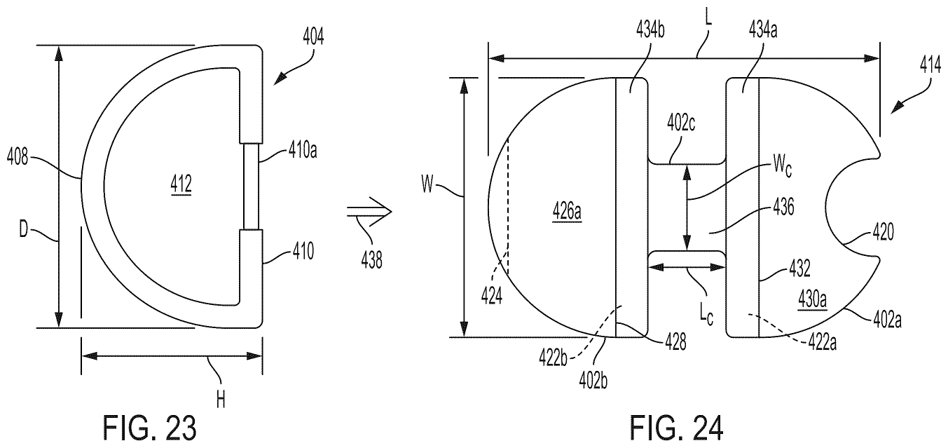

Handle 404 is formed as an open semicircular ring or loop in the embodiment shown in FIG. 23. Handle 404 comprises a curved portion 408 traversing an arc of approximately 180 degrees, the arcuate ends of which are joined by a straight connecting portion 410. Curved portion 408 and straight connecting portion 410 define a fully enclosed opening 412 that is sized and configured to allow for the insertion of a practitioner's fingers so as to be hand held. Connecting portion 410 has a centrally located section 410a of reduced dimension that serves as a pin that is rotatable within hinge 406, as will be described. In a particular arrangement, curved portion 408 and straight connecting portion 410 are each formed to have a circular cross-section. Handle 404 may be formed of thermoplastic polyurethane as a one-piece integral component in a molding process, although other suitable materials may be used. Other processes, such as machining, may also be used to form handle 404. Handle 404 is formed to be more rigid than anchor 402, although it may have some pliability when force is applied by a practitioner. In a particular size, handle 404 may have a semicircular diameter, D of 4.00 inches and a height, H of 3.00 inches. It should be understood that handle 404 may be formed to have other dimensions and may also have a shape other that semicircular, such as circular, or rectangular. Further, in some instances opening 412 need not be fully enclosed providing that there is sufficient structure for manually grasping handle 404.

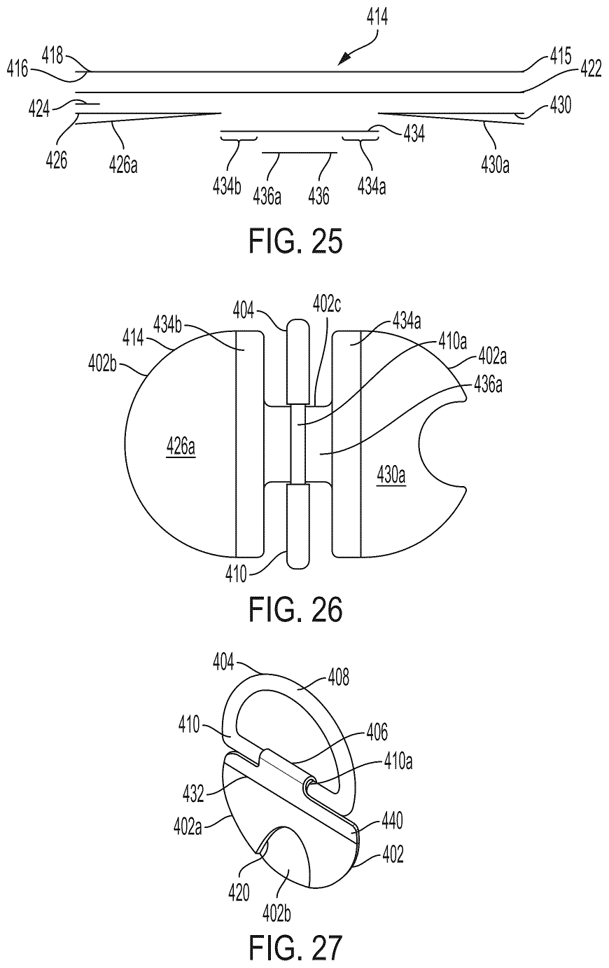

Turning now to FIGS. 24 and 25, details of the formation of anchor 402 are described. Anchor 402 is formed from an elongate base 414 that comprises a thin, flexible one-piece sheet 415 that is a substantially flat. Sheet 415 has a bottom surface 416, a top surface 418, and includes the first and second opposing anchor portions 402a and 402b, and a connecting portion 402c that joins anchor portions 402a and 402b. In one arrangement, anchor portions 402a and 402b are each shaped in the form of a semicircle. Connecting portion 402c has a width, Wc that is less than the diameters of anchor portions 402a and 402b, and a length, Lc between anchor portions 402a and 402b. A cutout 420 may be formed into the periphery of one of the anchor portions 402a or 402b to keep an area of the patient's skin, such as the navel area, open upon attachment of device 400 to the patient. As such, the provision of cutout 420 allows lifting device 400 to be applied without interfering with instruments that may be inserted through the patient's navel.

Sheet 415 provides high strength and little stretch under a tensile force and in one arrangement comprises polypropylene, colored white, although other suitable materials such as polyurethane, and colors may also be used. The semicircular diameters of anchor portions 402a and 402b may be substantially the same as the diameter, D of handle 404, establishing an overall width, W of base 414. In a particular arrangement, width, W may be 4.00 inches. In this arrangement, base 414 may have an overall length, L of approximately 6.26 inches, as shown in FIG. 24, width, Wc of connecting portion 402c may be 1.325 inches, the length, Lc of connecting portion 402c may be 1.25 inches, and the thickness of sheet 415 may be approximately 0.003 to 0.100 inches. It should be understood that base 414 may be formed to have other dimensions and that anchor portions 402a and 402b may have shapes other that semicircles.

A layer of adhesive 422 is included on the bottom surface 416 of sheet 415. In the arrangement shown, adhesive 422 extends along the entire length, L of base 414, including the bottom surfaces of opposing anchor portions 402a and 402b, as well as on the bottom surface of connecting portion 402c. In some instances, adhesive 422 may be included on only the bottom surfaces of opposing anchor portions 402a and 402b. Adhesive layer 422 in one arrangement, is the same as adhesive layer 26, described above, that is applied to the lower surface 24 of base 20. In other instances, the strength of adhesive 422 is preferably stronger than that of adhesive layer 26, and may comprise high initial tact, extended wear, acrylic adhesive.

A thin polymer film 424 similar to film 220 described above may be attached to adhesive layer 422, for example at the one end of base 414 as shown in FIGS. 24 and 25 to form a chordal dead zone on anchor portion 402b that will not attach to the patient's skin and that will assist the surgeon in the removal of tissue lifting device 400 upon completion of the procedure, as will be explained. Film 424 may be formed of the same material as sheet 415, or other suitable material, with a thickness in the range of 0.001 to 0.200 inches. In one arrangement, film 424 may extend about 0.250 inches from the end of base 414 toward the opposite end. The purpose of the dead zone is to prevent the surgeon's gloves from sticking to the base 414 while he/she removes the lifting device 400 from the patient after the procedure is completed and serves as a starting point for device removal.

A shown in FIG. 25, a removable liner 426 may cover adhesive layer 422 on the bottom surface 416 of anchor portion 402b. Liner 426 may have a tab 426a to facilitate removal from adhesive 422 for assembly, as will be described. Liner 426 is configured to cover all of adhesive 422 on the bottom surface of anchor portion 402b from the axial end to a fold line 428, leaving an exposed strip 422b of adhesive 422 that extends laterally across anchor portion 402b adjacent connecting portion 402c, as shown in FIGS. 24 and 25. At the opposite end of sheet 415, a removable liner 430 may cover adhesive layer 422 on the bottom surface of anchor portion 402a. Liner 430 may have a tab 430a to facilitate removal from adhesive 422 for assembly, as will be described. Liner 430 is configured to cover all of adhesive 422 on the bottom surface of anchor portion 402a from the axial end to a fold line 432, leaving an exposed strip 422a of adhesive 422 that extends laterally across anchor portion 402a adjacent connecting portion 402c, as shown in FIGS. 24 and 25. In one arrangement, liners 426a and 430a are silicone-coated paper with silicone on the side that contacts adhesive 422 and non-coated on the opposite side.

Adhesive strips 422a and 422b on anchor portions 402a and 402b form attachment zones for the formation of tissue lifting device 400, as will be described. In one arrangement, the portion of adhesive 422 between fold lines 428 and 432 including adhesive strips 422a and 422b as well as connecting portion 402c is covered with an additional separate layer of adhesive 434. Adhesive 434 may have stronger adhesive characteristics than adhesive 422 and that adheres well to itself. In some instances, adhesive 434 may comprise same material as adhesive 422, or may not be used at all. Where additional adhesive 434 is used, a deadener 436 may be applied to cover selective portions of adhesive 434. Adhesive deadener 436 extends substantially the length Lc and width Wc of connecting portion 402c without covering the bottom surface of anchor portions 402a and 402b. As such, adhesive attachment zones 434a and 434b are formed coincident with attachment zones 422a and 422b. Deadener 436, which may be formed of the same material as sheet 415, provides a non-adhesive interior surface 436a of hinge 406 within which handle centrally located section 410a may freely move, as will be described. Adhesive deadener 436 may also be a non-adhesive spray or coating applied to adhesive layer 434. In an arrangement where additional adhesive 434 is not used, deadener 436 is applied directly to adhesive 422, and in such instance, adhesive attachment zones 422a and 422b are used in the formation of hinge 406.

Referring now also to FIGS. 26-28, the formation of lifting device 400 is described. One end of base 414, such as at anchor portion 402b, is inserted through opening 412 of handle 402 in an orientation as schematically shown by arrow 438, until centrally located section 410a of handle connecting portion 410 is juxtaposed over non-adhesive surface 436a of deadener 436, as illustrated in FIG. 26. As described above, section 410a serves as a pin within hinge 406. At this point, anchor portions 402a and 402b are folded over pin 410a toward each other until anchor portions 402a and 402b are substantially parallel and face each other. In this position, adhesive attachment zones 434a and 434b, which are also aligned and facing each other, are adhesively securely joined. With such attachment, a handle support 440 is formed across the width of anchor 402 that includes those portions of anchor portions 402a and 402b comprising anchor zones 434a and 434b. Such attachment further forms hinge 406 wherein non-adhesive surface 436a surrounds pin 410a, as shown in FIG. 27. In this position, anchor portions 402a and 402b are parallel and face each other with liners 426 and 430 preventing sticking to each other. Anchor portions 402a and 402b are also parallel to handle support 440 and to handle 404.

Once device 400 is formed, handle 404 may freely rotate within hinge 406 relative to anchor 402 about an arc of approximately 360 degrees. As such, in one position, handle 404 may be positioned until it is facing and substantially parallel to anchor portion 402a, as depicted in FIG. 29. Handle may then be rotatably maneuvered in hinge 406 in the reverse direction until it is facing and substantially parallel to opposite anchor portion 402b.

Lifting device 400 may be packaged in a tray or container 500 for shipment to a hospital or other surgical facility. For shipment, lifting device 400 may be folded to the position shown in FIG. 29, where lifting device 400 is reduced to its minimum size with the anchor portions 402a and 402b being in contact or in closely spaced facing relation and substantially parallel to handle support 440. In such position, handle 404 lies closely adjacently over and substantially parallel to one of the two anchors portions 402a or 402b. Tray 500 may be clear plastic and packaged with one of more lifting devices 400 folded as shown in FIG. 29. Once one or more devices 400 are packaged in tray 500, those devices 400 and tray 500 may be sterilized together for shipment.

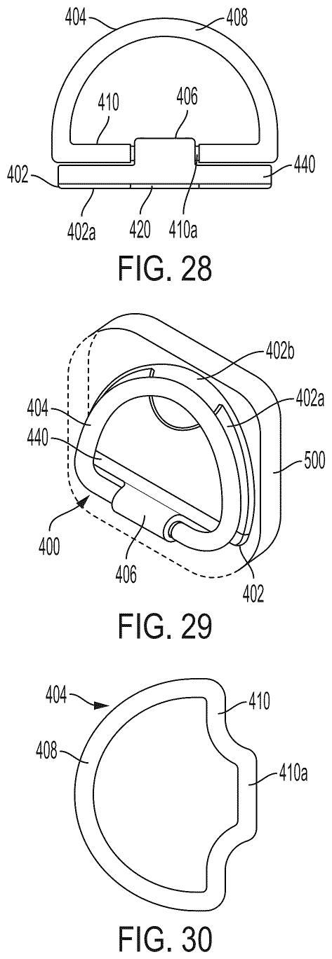

In use, lifting device 400 may be removed from tray 500 or other package at a surgical setting. Anchor portions 402a a and 402b may be folded away from each other along fold lines 428 and 432 to project outwardly transversely from handle support 440, as depicted in FIG. 22. In this position, as also shown in FIG. 28, anchor portions 402a and 402b are generally perpendicular to handle support 440 and lie in a common plane. Freely movable handle 404 may be rotated anywhere from parallel to and facing anchor portion 402a to a position parallel to and facing anchor portion 402b. In the position shown in FIGS. 22 and 28, handle 404 is substantially parallel to handle support 440 and perpendicular to the plane formed by anchor portions 402a and 402b. Device 400 may be attached to a selected area of the patient's skin by removing one of liners 426 or 430 by pulling tabs 426a and 430a to expose adhesive layer 422 once a respective liner is removed. For example, if liner 426 is removed first, anchor portion 402b is then adhesively attached to the skin of the patient. Upon adhesive attachment of anchor portion 402b, liner 430 is then removed and anchor portion 402a is adhesively attached to the patient's skin completing the process of attaching device 400 to the skin of the patient. If the attachment is near the patient's navel, the cutout portion 420 may be positioned such that the device 400 will not be adhered to the patient's navel. Further, as a result of the flexibility and thickness of anchor portions 402a and 402b, the anchor 402 of the lifting device 400 may readily follow the contours of the skin of the patient's abdomen.

Handle 404 is sufficiently sized and configured to allow the surgeon to slide his/her fingers of one hand into the opening 412 of handle 404 for grasping. Manual upward force is applied to anchor portions 402a and 402b with the load being transferred outwardly through hinge 406 and handle support 440 from the center of device 400. This load transfer allows handle 404 to be pulled outwardly from the patient and provide a sufficient force to lift the patient's skin. The surgeon may then use a Veress needle 18, a trocar or other instruments to gain access into the expanded abdominal cavity for surgery. Upon completion of use, lifting device 400 may be removed from the skin of the patient by the surgeon slipping his/her fingers beneath film 424 on the perimeter of device 400, which is not adhered to the patient, so as to grab and peel device 400 from the patient. Inasmuch as lifting device 400 is intended as a disposable device for single use, upon removal lifting device 400 may be suitably discarded.

It can now be appreciated that the inventive lifting device described herein is a hand-held device that is relatively low cost, effective, easy to use, and particularly useful for lifting abdominal tissue in laparoscopic surgery without attachment to a frame, vacuum source or other static apparatus. It should also be appreciated that variations may be made to lifting device described herein without departing from the contemplated scope of the invention. For example, handle 404 may be formed such that the centrally located section 410a that serves as a hinge pin is axially offset relative to connecting portion 410, thereby providing more height to handle 404. It should further be understood that the subject lifting device may also be used to lift or otherwise manipulate tissue of a patient in medical procedures other than laparoscopic surgery. Accordingly, only the preferred embodiments have been presented herein and all changes, modifications and further applications that come within the spirit of the invention are desired to be protected.

What is claimed is:

1. A hand held tissue lifting device, comprising:
an anchor including an upper surface and a lower surface, and a layer of adhesive on the lower surface for attachment to skin of a patient; and
a handle that is sized and configured to be hand held by a user, the handle being movably attached to the upper surface of the anchor.

2. The hand held tissue lifting device of claim 1, wherein the anchor and the handle are formed as an integral one piece device.

3. The hand held tissue lifting device of claim 1, wherein the anchor and the handle are separate components attachable to each other to form the tissue lifting device.

4. The hand held tissue lifting device of claim 3, wherein the handle defines an opening that is sized and configured for insertion of a user's fingers such that the tissue lifting device may be hand held.

5. The hand held tissue lifting device of claim 4, wherein the opening is fully enclosed.

6. The hand held tissue lifting device of claim 5, wherein the fully enclosed opening is defined by a semicircular ring or loop.

7. The hand held tissue lifting device of claim 3, wherein the handle is formed of a material that is more rigid than the material of the anchor.

8. The hand held tissue lifting device of claim 7, wherein the anchor comprises a sheet of flexible polymer material that provides relatively high strength and little stretch under a tensile force.

9. The hand held tissue lifting device of claim 8, wherein the sheet of flexible polymer material has a thickness that allows the anchor to follow contours of a patient's skin.

10. The hand held tissue lifting device of claim 9, wherein the sheet of flexible polymer material has a thickness of 0.003 to 0.100 inches.

11. The hand held tissue lifting device of claim 3, wherein the handle is attached to the anchor at a hinge that allows free rotation of the handle relative to the anchor.

12. The hand held tissue lifting device of claim 1, wherein the layer of adhesive on the lower surface of the anchor is covered by a removable liner.

13. The hand held tissue lifting device of claim 1, wherein a non-adhesive dead zone is formed on a least a portion of a periphery of the anchor to assist a user in removal of the hand held tissue lifting device from the patient.

14. A hand held tissue lifting device, comprising:
a flexible anchor comprising a first anchor portion and a second anchor portion, a first layer of adhesive on a lower surface of the first anchor portion for attachment to skin of a patient and a second layer of adhesive on a lower portion of the second anchor portion for attachment to skin of a patient;
a handle support connecting the first anchor portion and the second anchor portion;
a first fold line adjacent the handle support, the first anchor portion being foldable about the first fold line;
a second fold line adjacent to the handle support, the second anchor portion being foldable about the second fold line; and
a handle sized and configured to be hand held, the handle being attachable to the handle support for movement relative to the first anchor portion and the second anchor portion.

15. The hand held tissue lifting device of claim 14, wherein the first anchor portion and the second anchor portion are folded about the respective fold lines such that the first anchor portion and the second anchor portion are substantially parallel to and face each other and are substantially parallel to the handle support.

16. The hand held tissue lifting device of claim 14, wherein the handle support includes a hinge, and wherein the handle is rotatably attached to the handle support within the hinge.

17. The hand held tissue lifting device of claim 16, wherein the handle is rotatable within the hinge relative to the first and second anchor portions about an arc of approximately 360 degrees.

18. The hand held tissue lifting device of claim 17, wherein the handle lies substantially parallel to and in closely spaced facing relation to the first anchor portion or the second anchor portion.

19. The hand held tissue lifting device of claim 14, wherein the first anchor portion and the second anchor portion are folded about the respective fold lines such that the first anchor portion and the second anchor portion are substantially parallel to each other and substantially perpendicular the handle support, the first anchor portion and the second anchor portion lying in a common plane.

20. The hand held tissue lifting device of claim 14, wherein the handle defines an opening that is sized and configured for insertion of a user's fingers such that the handle may be hand held.

21. The hand held tissue lifting device of claim 20, wherein the opening is fully enclosed.

22. The hand held tissue lifting device of claim 21, wherein the fully enclosed opening is defined by a semicircular ring or loop.

23. The hand held tissue lifting device of claim 14, wherein each of the first anchor portion and the second anchor portion comprises a sheet of polymer material that provides relatively high strength and little stretch under a tensile force.

24. The hand held tissue lifting device of claim 14, wherein the first layer of adhesive on the lower surface of the first anchor portion and the second layer of adhesive on the lower surface of the second anchor portion are each covered by a removable liner.

25. The hand held tissue lifting device of claim 14, wherein a cutout is formed into a periphery of at least one of the first anchor portion or the second anchor portion.

26. The hand held tissue lifting device of claim 14, wherein a non-adhesive dead zone is formed on a portion of a periphery of one of the first anchor portion or the second anchor portion to assist a user in removal of the hand held tissue lifting device from the patient.

27. A container, comprising:
one or more hand held tissue lifting devices of claim 14; and
a tray within which the one or more hand held tissue lifting devices are contained.

28. The container of claim 27, where the tray and one or more of the hand held tissue lifting devices are sterilized.

29. A hand held tissue lifting device formed by a process comprising the steps of:
providing an elongate base having a first end and an opposing second end, an upper surface, a lower surface, a first anchor portion adjacent the first end and a second anchor portion adjacent the second end, a connecting portion disposed between and joining the first anchor portion and the second anchor portion, a first attachment zone between the first anchor portion and the connecting portion, a first fold line between the first anchor portion and the first attachment zone, a second attachment zone between the second anchor portion and the connecting portion, a second fold line between the second anchor portion and the second anchor portion, a first layer of adhesive on a lower surface of the first attachment zone, a second layer of adhesive on a lower surface of the second attachment zone, and a non-adhesive surface on a lower surface of the connecting portion;
providing a handle sized and configured to be hand held by a user, the handle having a section defining a hinge pin;
placing the handle in juxtaposition to the elongate base with the hinge pin in alignment with the non-adhesive surface of the connecting portion; and
folding the connecting portion over the hinge pin to move the first and second anchor portions toward each other to place the first attachment zone and the second attachment zone in alignment, and then joining the first layer of adhesive of the first attachment zone to the second layer of adhesive of the second attachment zone to thereby form a hinge about the hinge pin within which the handle may freely rotate.

30. The hand held tissue lifting device of claim 29, wherein the first anchor portion comprises a first adhesive on a lower surface and the second anchor portion comprises a second adhesive on a lower surface, and wherein each of the first adhesive and the second adhesive is covered by a removable liner.
